(19) United States
(12) Patent Application Publication
(10) Pub. No.: US 2026/0092915 A1
(43) Pub. Date: Apr. 2, 2026

(54) TEST DEVICE AND METHOD FOR ANALYZING BIOLOGICAL SAMPLES USING BIFURCATED FLUIDIC PATHWAYS

(71) Applicant: Inito Health Inc., Houston, TX (US)

(72) Inventors: Varun AKUR VENKATESAN, Bangalore (IN); Siddharth PATTNAIK, Bangalore (IN); Dipankar DAS, Bangalore (IN)

(21) Appl. No.: 19/411,423

(22) Filed: Dec. 8, 2025

Related U.S. Application Data
(63) Continuation-in-part of application No. 17/620,761, now Pat. No. 12,517,116.

(30) Foreign Application Priority Data
Jun. 20, 2019 (IN) 201911024493

(51) Int. Cl. G01N 33/543; B01L 3/00

(52) U.S. Cl. CPC G01N 33/54389; B01L 3/5023; B01L 2300/0825; B01L 2300/0864

(57) ABSTRACT

The present disclosure provides a method for detecting one or more analytes in a biological sample using a test device. The method includes applying the biological sample to a sample-introduction region to obtain a flowing sample along a primary fluidic pathway. The obtained flowing sample may bifurcate at one or more bifurcation nodes into at least two laterally directed fluid branches that advance toward one or more detection pads arranged in an array along the primary fluidic pathway. The obtained flowing sample may interact with one or more biochemical agents present at the one or more detection pads to generate a signal indicative of the presence of the one or more analytes in the biological sample.

### CROSS-REFERENCE TO RELATED APPLICATIONS

This application is a continuation-in-part of U.S. patent application Ser. No. 17/620,761, filed 2021 Dec. 20, which published as U.S. Patent Application Publication No.2022/0341919 A1 on Oct. 27, 2022, the entire disclosure of which is incorporated herein by reference.

### FIELD OF INVENTION

The present disclosure relates to test devices for analyzing biological samples, and more particularly to a test device with bifurcated fluidic pathways that direct sample flow to multiple detection pads for detecting and quantifying analytes in biological samples.

### BACKGROUND

Urine dipstick tests are widely used diagnostic tools for detecting various analytes such as glucose, proteins, ketones, blood, and other biomarkers in biological samples. Traditional urine dipsticks typically consist of a plastic or paper strip with one or more reactive pads that change color upon contact with specific analytes in the sample. While these devices offer a quick and cost-effective means of screening, they suffer from several technical and practical limitations that affect their accuracy, reliability, and user convenience.

Conventional dipsticks require full immersion of the reactive pad into the urine sample for accurate results. Partial dipping may lead to incomplete reagent activation and false readings. Additionally, a sufficient volume of sample is needed in a container to allow full immersion, making testing difficult or unreliable when only small sample volumes are available. The dipping process also introduces a risk of cross-contamination between the stick and the sample, particularly in multi-test scenarios or clinical settings. Furthermore, dipsticks often require precise timing for immersion and reading, and deviations from recommended protocols can cause inaccurate color development and misinterpretation of results. The linear arrangement of reactive pads on traditional dipsticks means that improper dipping angle or incomplete contact can result in uneven reagent exposure across different detection zones.

From a user convenience perspective, the available dipsticks as of now involve several challenges. Users must collect urine in a separate container and then dip the stick into the sample, which is inconvenient and unhygienic, especially in home-testing scenarios. The process of transferring urine into a container and dipping the stick increases the likelihood of spills and mess, making the experience unpleasant. Carrying a container for sample collection is impractical for on-the-go or emergency testing situations. Non-professional users often fail to dip the stick fully or maintain correct timing, leading to unreliable results and increased user error. Additionally, both the container and the dipped stick require careful disposal, adding complexity to the testing process. There remains a need for improved test devices and methods that address these limitations while providing accurate and reliable detection of analytes in biological samples.

### SUMMARY OF THE INVENTION

In some aspects of the present disclosure, a method for detecting one or more analytes in a biological sample includes applying the biological sample to a sample-introduction region of a test device to obtain a flowing sample along a primary fluidic pathway. The method further includes enabling bifurcation of the obtained flowing sample at one or more bifurcation nodes disposed along the primary fluidic pathway into at least two laterally directed fluid branches to flow toward one or more detection pads arranged in an array along the primary fluidic pathway, such that the flowing sample interacts with one or more biochemical agents present at the detection pads to generate and detect a signal indicative of the presence of the one or more analytes.

In some aspects, the test device includes a lateral flow assay.

In some aspects, the configuration of the primary fluidic pathway includes directing the applied biological sample to flow vertically along the primary fluidic pathway.

In some aspects, the configuration of the primary fluidic pathway includes directing the applied biological sample to flow substantially perpendicular along the primary fluidic pathway.

In some aspects, the configuration of the primary fluidic pathway includes directing the applied biological sample to flow angularly along the primary fluidic pathway.

In some aspects, the flowing sample progresses along the primary fluidic pathway in a first direction and subsequently progresses along the laterally directed fluid branches in a second direction transverse to the first direction.

In some aspects, the flowing sample is divided at each bifurcation node into first and second lateral branches positioned on opposite sides of the primary fluidic pathway.

In some aspects, each of the one or more detection pads include receiving the obtained flowing sample from two laterally opposed branches.

In some aspects, the signal includes representing and indicating concentration or level of the one or more analytes present in the biological sample.

In some aspects, the signal includes colorimetric, fluorescent, chemiluminescent, radiometric, or electrochemical.

In some aspects, the biological sample includes urine, semen, sperm, blood, tears, and saliva.

In some aspects of the present disclosure, a method for improving quantification of a biological sample in a test device includes applying the biological sample to a sample-introduction region of the test device to obtain a flowing sample along a primary fluidic pathway. The method further includes enabling bifurcation of the obtained flowing sample at one or more bifurcation nodes into at least two laterally directed fluid branches, facilitating the flowing sample to pass through at least two successive bifurcation levels prior to reaching one or more detection pads, allowing the flowing sample to contact the detection pads comprising one or more biochemical agents configured to generate and detect signal, and quantifying the biological sample by measuring the signal to obtain an improved quantification output.

In some aspects, a test device for detecting one or more analytes in a biological sample includes a sample-introduction region configured to receive the biological sample, a primary fluidic pathway extending in a first direction, one or more bifurcation nodes configured to divide the flowing sample into first and second laterally extending branches positioned on opposite sides of the primary fluidic pathway, and one or more detection pads arranged in an array along the primary fluidic pathway, each detection pad being fluidically connected to a respective pair of laterally extending branches and enabling the sample to flow along the primary fluidic pathway and subsequently outward and inward toward the detection pads.

In some aspects, the test device includes a lateral flow assay device. In some aspects, the configuration of the primary fluidic pathway includes directing the received biological sample to flow vertically, substantially perpendicular, or angularly along the primary fluidic pathway. In some aspects, the array of detection pads includes a spaced tiered arrangement along the primary fluidic pathway. In some aspects, each detection pad includes one or more biochemical agents configured to generate signal that enables quantification of analyte level across the array. In some aspects, each detection pad is positioned along a central axis relative to the pair of laterally extending branches. In some aspects, the biological sample includes urine, semen, sperm, blood, tears, and saliva.

In some aspects, a test device for improving quantification of a biological sample includes a sample-introduction region, a primary fluidic pathway, one or more bifurcation nodes configured to divide the flowing sample into first and second laterally extending branches, and one or more detection pads arranged in a spaced tiered arrangement along the primary fluidic pathway, each detection pad being fluidically connected to a respective pair of laterally extending branches and enabling the sample to flow along the primary fluidic pathway and subsequently outward and inward toward the detection pads.

### BRIEF DESCRIPTION OF FIGURES

Non-limiting and non-exhaustive examples are described with reference to the following figures.

FIG. 1 is an isometric view of a test strip assembly 101 of the device for analysis of fluids, according to an embodiment herein;

FIG. 2 is a top view of the test strip assembly 101, according to an embodiment herein;

FIG. 3 is an isometric view of the test strip assembly 101 showing a minimum of three detection labels, according to an embodiment herein;

FIG. 4 is a top view of the device for the analysis of fluids showing two test strips assemblies of FIG. 1-3, placed parallel to each other, according to an embodiment herein;

FIG. 5 is a top view of the device of FIG. 4 inserted into a housing, according to an embodiment herein;

FIG. 6 is a side view of the device of FIG. 4, according to an embodiment herein; and

FIG. 7 shows the flow of the analyte sample fluid through capillary action in the test strop of FIG. 1, according to an embodiment herein.

FIG. 8 is a flowchart illustrating a method for detecting one or more analytes in a biological sample, according to an embodiment herein.

FIG. 9 illustrates a test device for detecting one or more analytes in a biological sample, according to an embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

As mentioned, there remains a need for a diagnostic strip and device for analysing bodily fluids that require only partial exposure of the strip and device into the bodily fluid sample. The embodiments herein achieve this by providing a strip and a device incorporating the strip is required to be dipped at the tip into the bodily fluid sample for detecting the presence or absence of one or more analytes in the bodily fluid sample.

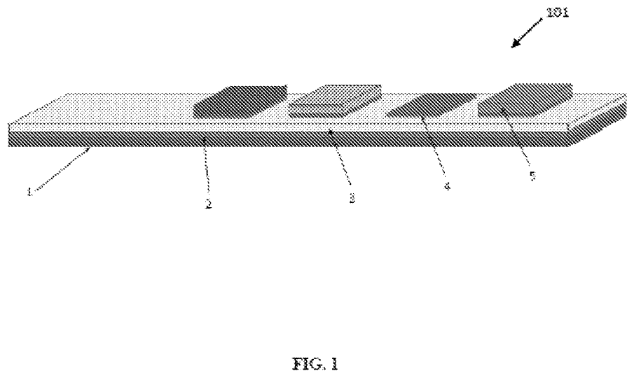

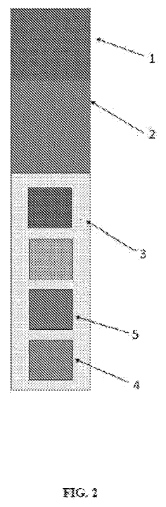

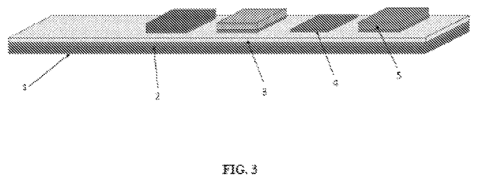

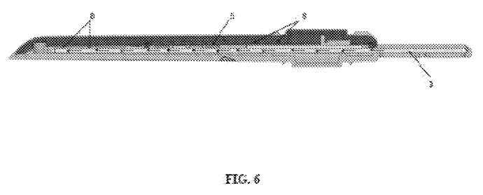

Referring now to the drawings, and more particularly to FIGS. 1 through 7 <incorporated from U.S. Patent Application Publication No.2022/0341919A1 entirety>, where similar reference characters denote corresponding features consistently throughout the figures, there are shown preferred embodiments.

The term “bodily fluid” or “bodily fluid sample” or “biological sample” refers to the fluids in or on a human or animal body. The examples include sweat, urine, blood, blood serum, semen, breast milk, saliva, blood plasma, tears, mucus, cerebrospinal fluids, saliva, amniotic fluid, vaginal lubrication fluids, pus, lymph, bile, synovial fluid, aqueous humour, phlegm, gastric acid, pre-ejaculate, colostrum and other such fluids related to animals or humans. The bodily fluid may also include the bodily matter that has been liquified by mixing in a solvent such as water. The phrases “bodily fluid”, “bodily fluid sample”, “biological sample” and “sample” are used interchangeably across the present specification.

The term “analyte” or “one or more analytes” refers to hormones, ions, proteins, lipids, sugar, oxygen, antibodies, enzymes, carbohydrates, virus, bacteria or any other foreign particles, metabolites that may be detected and analysed, qualitatively or quantitatively, to determine the state of health or general well-being of an animal or a human

The following description sets forth exemplary aspects of the present disclosure. It should be recognized, however, that such description is not intended as a limitation on the scope of the present disclosure. Rather, the description also encompasses combinations and modifications to those exemplary aspects described herein.

The terms ‘user,’ ‘patient,’ ‘body,’ and ‘subject’ may refer to similar meaning/interpretation and may be interchangeably.

The terms “test device” and “device” are interchangeably used throughout the specification.

The terms “bodily fluid”, “bodily fluid sample”, “biological sample” and “sample” are used interchangeably across the present specification.

As used herein, the term “primary fluidic pathway” (represented as arrows to illustrate the flow direction) refers to a main channel or conduit along which a biological sample flows after being applied to a sample-introduction region. The primary fluidic pathway may extend in a first direction and may serve as a central flow path from which lateral branches diverge. The first direction may refer to the primary flow axis along which sample travels through the test device, where the first direction may be vertical (extending downward from top to bottom), horizontal (extending laterally from one side to another), perpendicular (extending at approximately 90 degrees relative to a reference plane), or angular (extending at an oblique angle relative to a reference plane). The primary fluidic pathway may provide a controlled route for sample transport through the test device.

As used herein, the term “one or more bifurcation nodes” refers to specific locations or structures along the primary fluidic pathway where a flowing sample may be divided or split into multiple flow paths. The one or more bifurcation nodes may direct sample portions into laterally directed branches. Each bifurcation node of the one or more bifurcation nodes may be configured to promote controlled sample division, allowing for systematic distribution of sample aliquots to different regions of the test device.

As used herein, the term “laterally extending branches” or “laterally directed fluid branches” refers to secondary flow channels that may extend from one or more bifurcation nodes in a direction transverse or perpendicular to the primary fluidic pathway. The laterally extending branches may direct portions of a flowing sample outward from the primary pathway and subsequently inward toward one or more detection pads. The laterally extending branches may provide a means for delivering biological sample to multiple detection zones while maintaining controlled flow characteristics. The term “first and second laterally extending branches” refers to a pair of branches that extend from a bifurcation node on opposite sides of the primary fluidic pathway, where the first laterally extending branch may extend to one side (such as the left side) of the primary fluidic pathway and the second laterally extending branch may extend to the opposite side (such as the right side) of the primary pathway, creating a symmetric branching pattern.

As used herein, the term “one or more detection pads” refers to regions or zones within the test device where analyte detection and quantification occur. The one or more detection pads may contain one or more biochemical agents configured to interact with one or more analytes present in a biological sample. The one or more detection pads may generate a signal upon analyte interaction, where the signal intensity may correlate with analyte concentration or level to enable quantitative measurements. Aspects of the present disclosure are intended to include or otherwise cover any number of detection pads, without deviating from the scope of the present disclosure.

As used herein, the term “one or more biochemical agents” refers to molecules or compounds capable of specifically interacting with or binding to one or more analytes. The one or more biochemical agents may include antibodies, enzymes, aptamers, nucleic acid probes, or other binding molecules. The one or more biochemical agents may be immobilized on or within the one or more detection pads. The one or more biochemical agents may be selected from a group including, but not limited to, enzyme labels such as horseradish peroxidase (HRP), alkaline phosphatase (AP), glucose oxidase, or β-galactosidase that catalyze colorimetric or chemiluminescent reactions, fluorescent labels including fluorescein isothiocyanate (FITC), rhodamine derivatives, Alexa Fluor dyes, or cyanine dyes for optical detection, quantum dots providing enhanced photostability and narrow emission spectra, or luminescent nanoparticles such as europium chelates or terbium complexes for time-resolved fluorescence detection. Aspects of the present disclosure are intended to include or otherwise cover any type of biochemical agents, without deviating from the scope of the present disclosure.

As used herein, the term “signal” refers to a measurable output generated as a result of interaction between the one or more analytes and the one or more biochemical agents. The signal may be colorimetric, fluorescent, chemiluminescent, radiometric, or electrochemical. The signal may represent and indicate the concentration or level of the one or more analytes present in a sample, where signal intensity may be directly or inversely proportional to analyte concentration depending on the detection mechanism employed.

As used herein, the term “bifurcation level” refers to a stage or tier in a fluidic network where sample flow may be divided at one or more bifurcation nodes positioned at approximately the same distance along the primary fluidic pathway. Multiple successive bifurcation levels may enable controlled sample distribution to increasing numbers of the one or more detection pads.

As used herein, the term “quantification” refers to the process of determining the concentration, amount, or level of one or more analytes present in a biological sample. Quantification may be accomplished through measurement of signal intensity, color development, fluorescence emission, or other quantifiable parameters that correlate with analyte concentration or level. Quantification may provide numerical concentration values or may provide semi-quantitative results indicating concentration ranges.

As used herein, the term “optical analysis system” refers to devices or instruments capable of measuring optical signals generated at one or more detection pads. Optical analysis systems may include optical scanners, smartphone cameras, dedicated imaging devices, spectrophotometers, fluorescence readers, handheld imaging sensors, portable readers, handheld analyzers, or other optical measurement instruments. Optical analysis systems may capture images or measure light intensity to quantify analyte concentrations based on signal characteristics. Aspects of the present disclosure are intended to include or otherwise cover any type of optical analysis system or imaging sensor, without deviating from the scope of the present disclosure.

As used herein, the term “spaced tiered arrangement” refers to a configuration where one or more detection pads may be positioned at different vertical or horizontal levels along the primary fluidic pathway, where each detection pad may be separated from adjacent detection pads by a defined spacing. The spaced tiered arrangement may enable sequential or simultaneous sample delivery to multiple detection pads while maintaining controlled flow characteristics and preventing cross-contamination between detection zones.

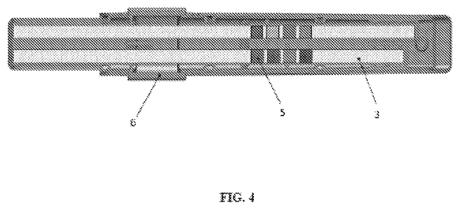

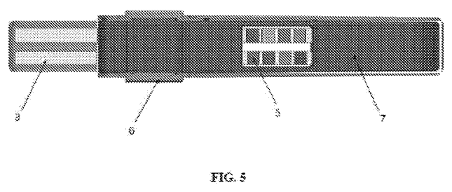

FIG. 1-3 illustrates different views of a test strip assembly 101 of the device for analysis of fluids, according to an embodiment herein. The test strip assembly 101 includes a basal layer 1, a first adhesive layer 2, a porous membrane 3, a second adhesive layer 4, and a plurality of detection labels 5. In an embodiment, the first adhesive layer 2 is entirely present over the basal layer 1. In an embodiment, the porous membrane 3 is present over the first adhesive layer 2. The number of detection labels 5 are connected with the porous membrane 3 through the second adhesive layer 4. In a preferred embodiment, the porous membrane 3 is smaller or equal in dimension to the basal layer 1. In an embodiment, the detection labels 5 are smaller in dimension compared to the porous membrane 3 and vary in number on one test strip 101. The entire test strip assembly 101 is placed into a customized cassette/housing 6, as shown in FIG. 4-6, which is greater than or equal in length to the test strips assembly 101. The cassette/housing 6 has an opening. The opening is covered by a removable cap. The opening is designed to expose the zone containing the detection labels 5 on the test strip assembly 101 in order to be read by naked eyes or by a reader. In an embodiment, the housing 6 can house a number of test strips 101 to run several kind of tests parallelly and/or simultaneously using different bodily fluids at the same time. In an embodiment, the housing 6 houses the number of test strip assemblies 101 such that each of the test strip assembly is parallel to each other. FIG. 4 and FIG. 5 illustrates a housing 6 having two test strip assemblies 101 lying parallel to each other, within the housing. The housing, thus, can be adapted to include as many test strip assemblies 101 as possible to have multiple detection and/or diagnosis using different bodily fluids simultaneously at the same time.

According to an embodiment of the present invention, the basal layer 1 of the test strip assembly 101 is made up of resins, metal foils, or glass. The resins are selected from the group consisting of polyvinyl alcohol, polystyrene, polyvinyl chloride, polyester or a polyamide. The adhesive layers between the basal layer/sheet 1 and porous membrane 3, and between the porous membrane 3 and detection labels 5 is made up of fusion of adhesives like polyethylene or polyvinyl alcohol. The first adhesive layer 2 covers the entire top surface of the basal layer 1. The adhesive layer 2 needs to be hardened so that the abutting of basal sheet 1 and porous membrane 3 is firm. The second adhesive layer 4 covers the bottom surface of the detection label 5 entirely or enough firmly affix the detection label to the porous membrane 3. The material used as adhesive can be the same for both the adhesive layers.

The porous membrane 3 is made up of cellulose based polymers such as nitrocellulose membrane and nonwoven cellulose fibre membranes. Alternatively, blends of natural and synthetic polymers such as CytoSep is also used depending on the application. Glass fibres are also used as a material for the porous membrane layer. Typically, the thickness of the porous membrane should be between 15-200 microns and 40-80% of the surface should be porous. The membrane can be varied according to the type of metabolite to be analysed, within the given limits.

In a preferred embodiment, the porous membrane in the strip 101 is made up of several material. In a preferred embodiment, the porous membrane is made up of are either woven polymers, cellulose polymers, glass fibre polymers or mixed polymers that are a mixture of natural and synthetic polymers. The examples of woven polymers include cotton and nylon. The examples of cellulose polymer membranes include nitrocellulose membranes. The examples of mixed polymers include membrane materials such as CytoSep and Vivid Plasma Membrane. The pore sizes for the membranes typically range from 8-15 microns for all samples with very low viscosity such as urine or water. In an embodiment, the low viscosity fluids have viscosity less than 0.002 Pa·s. However, while working with highly viscous fluids such as whole blood, membranes with low pore size ranging from 0.1-5 microns are generally used. In an embodiment, the high viscosity fluids have viscosity higher than 0.002 Pa—s.

The detection labels 5 preferably includes an absorbent carrier such as filter paper impregnated with reagents that change colour upon chemical reaction with the metabolite. In an embodiment, a mix of detection reagents with neutral solid materials can also be used as detection labels. The reagents are immobilised on detection pads or labels 5 in a manner that avoids cross-contamination of reagents i.e. immobilized reagent doesn't flow along with the analyte in a bodily fluid. In a preferred embodiment, the absorption properties of all the detection pads or labels in the assay must be the same in order to maintain a uniform flow rate from start to end of the assay. In a preferred embodiment, the thickness of all detection pads or labels 5 is kept same and the distance between them is maintained in order to enable uniform availability of the analyte, in a bodily fluid, at different detection pads or labels 5.

The device and the test strip 101 assembly is required to be dipped partially into the bodily fluids sample meant for analysis such that only the tip of the device is required to be dipped.

In a preferred embodiment, the direction of flow of the liquid analyte or bodily fluid is lateral i.e. along the membrane 3, and the flow of the same analyte, after contacting with detection pads/labels 5, is vertical in the detection pads or labels 5.

The labels for bodily fluids may be for detecting glucose, ketones, uric acid, bilirubin, urobilinogen, pH and specific gravity. In an embodiment, a plurality of detection labels 5 may be arranged on the test strip 101. In an embodiment, the test strip 101 includes label for testing pH, which includes reagents such as methyl red, bromothymol blue and methanol or a mix of them. In another embodiment, the test strip 101 includes label for testing presence or absence of protein, which includes reagents such as sodium Sodium citrate, Citric acid, lauroylsarcosine, water, Magnesium sulfate, Tetrabromophenolphthalein ethyl ester and methanol or a mix of them. In yet another embodiment, the test strip 101 includes a label 105 for detection of urobilinogen in urine and the label includes reagents such as 4-cyclohexylaminobenzaldehyde, Oxalic acid, Methanol or a mix of them. Similarly, the label 105 for detecting glucose includes O-tolidine, Peroxidase, Glucose oxidase, Tartrazine, Ethanol (44%) or a mix of them. In still another embodiment, the test strip 101 includes a label 5 for detection of hydrogen peroxide via reagents that include Polyvinyl propionate dispersion, Phosphate buffer, Sodium alginate, Sodium lauryl sulfate, O-tolidine, Peroxidase and Methanol or a mix of them. Similarly, the presence of nitrates may be determined by the test strip 101 through the label 5 incorporating reagents that include sulphanilamide, a-naphthylamine, tartaric acid and methanol or a mix thereof.

The layer of porous cellulose polymer membrane in between the backing sheet and the detection labels allow the liquid analyte to travel from the point of contact with strip to the detection labels by capillary action. This ensures a controlled flow of the analyte to the detection labels which makes the analysis easier and more accurate. Furthermore, due to this porous membrane enabled capillary action, only the tip of the strip is required to be dipped in an analyte solution.

The plastic cassette/housing 6 and top cover 7 are made up of plastic materials such as polycarbonate plastics, acrylonitrile butadiene styrene, high density polyethylene, polystyrene and polypropylene. A combination of two or more plastics can also be used. Preferably, the same material is used for the plastic cassette and the cap.

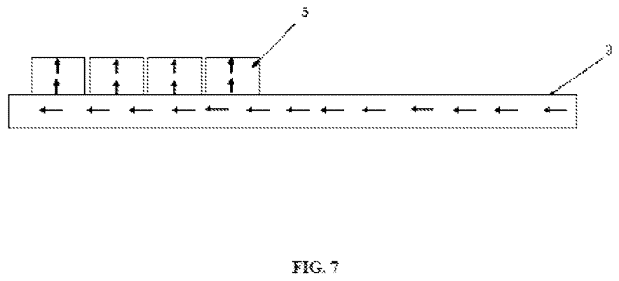

The assembly of the test strip 101 circumvents the need for dipping the entire strip into the liquid analyte. In addition, placing the test strip in a cassette aids easy handling. The user may hold the housing from one end and the open end of the test strip can be dipped into the analyte solution. Once the analyte reaches the detection layer by capillary action, it can access the detection layer preponderantly from beneath, wetting the detection label which gives a quick and readable colour reaction. FIG. 7 shows the flow of the analyte sample fluid through capillary action, according to an embodiment of the present invention. The plurality of openings 8 act as pinch points in the cassette for flow control of analyte sample. Thus, the test strip provides an easy and a more controlled way of detecting metabolites and metals in liquid analytes, which signifies greater consistency in the test results from one test to another.

In an embodiment, the device is adapted to be read by an optical device such that the labels 5 can be read using smartphone's or any handheld device's camera and light source to detect presence or absence as well as quantity of any constituent of any bodily fluid. In an embodiment, device is inserted into an optical device having a transparent optic defining an optical volume, a transparent optic having a first main face adapted for positioning the test strip assemble 101 for labels 5 to be imaged. The transparent optic is adapted to admit into the optical volume a light emitted by the light source for illuminating the labels 5 and wherein the transparent optic is adapted to admit the light having interacted with the labels 5, into the optical volume and turn the light inside the optical volume such that the light is internally reflected within the optical volume and exit the optical volume to be incident onto the camera. Alternatively, the device of the present embodiment can be inserted into a stand-alone or specialised optical readers or devices meant to detect presence or absence of an analyte in a bodily fluid.

In an embodiment, a method for detection of an analyte in the bodily fluid sample using a strip that is only required to be dipped partially into the bodily fluid sample is provided. The method includes dipping the device of FIG. 4, having the housing that carries or encloses the test strip assembly of FIG. 1 into the bodily fluid sample such that bodily fluid sample travels in a lateral direction in the porous membrane of the test strip assembly and the detection label, placed on the porous membrane, receives the bodily fluid such that in the detection label flow of the bodily fluid sample is in vertical direction. This is followed by subjecting the device into an adapter that can be adapted to or attached to a handheld camera device such as a smartphone, such that the detection labels are read by the camera, to detect the presence or absence of any analyte in the bodily fluid sample and convey the results of the test to a cloud server or locally.

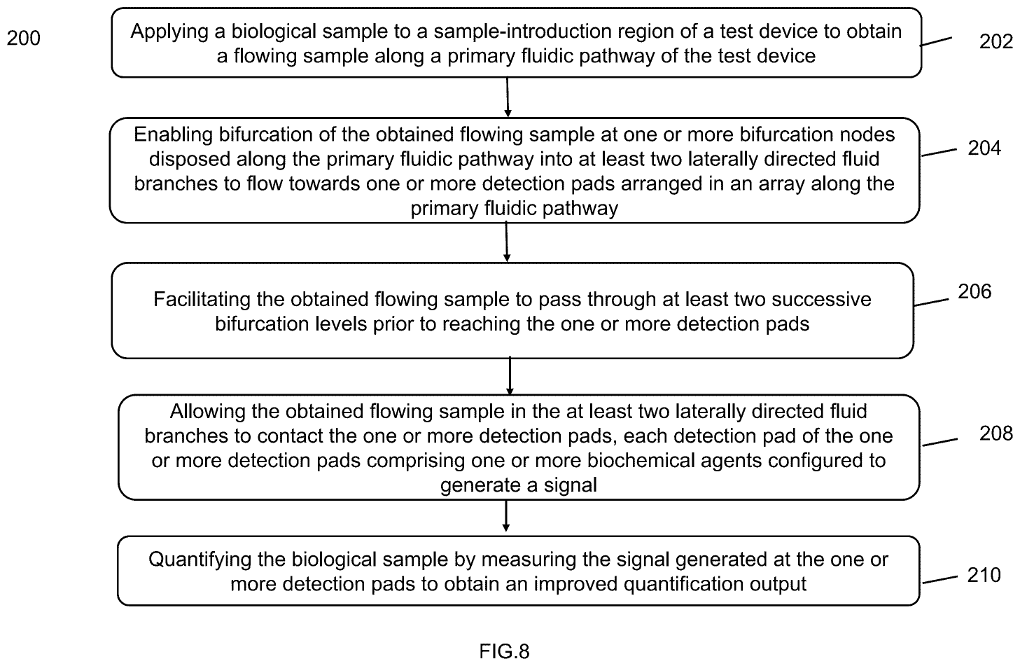

Referring to FIG. 8, a method 200 for detecting one or more analytes in the biological sample may provide a systematic approach for multiple analyte detection and quantification using the test device 300 with a specialized fluidic architecture. The method 200 may enable controlled sample distribution and analyte quantification through a series of coordinated steps that direct sample flow through bifurcating pathways to multiple detection zones. The method 200 may facilitate quantitative and qualitative analysis of biological samples by providing controlled sample delivery to the one or more detection pads 304A-N where specific interaction reactions occur and quantifiable signals are generated.

The method 200 may include applying 202 the biological sample to the sample-introduction region 302 of the test device 300 to obtain a flowing sample along a primary fluidic pathway of the test device 300. Applying 202 may be accomplished through various application techniques depending on the configuration of the sample-introduction region 302 and the nature of the biological sample. Applying 202 may involve dipping the test device 300 into a sample container, where the sample-introduction region 302 contacts the biological sample and draws sample into the device 300 through capillary action or other transport mechanisms. In some aspects, the applying 202 the biological sample may involve directly applying the biological sample to a well or reservoir formed in the sample-introduction region 302, where the biological sample may be deposited using a pipette, dropper, or other dispensing tool. Applying the biological sample 202 may also involve applying the biological sample to an absorbent pad that forms part of the sample-introduction region 302, where the pad may absorb and retain the sample before releasing the sample into the primary fluidic pathway. In some aspects, applying 202 the biological sample may involve stream application, where the sample may be applied as a continuous or intermittent stream onto the sample-introduction region 302. Aspects of the present disclosure are intended to include or otherwise cover any type of sample application technique, without deviating from the scope of the present disclosure.

In some aspects, the sample-introduction region 302 may include various configurations selected based on volume requirements and the intended application method. In some aspects, the sample-introduction region 302 may include a well for receiving larger sample volumes <not shown in fig>, where the well may have a defined capacity and may be shaped to facilitate sample retention and controlled release into the primary fluidic pathway. In some aspects, the sample-introduction region 302 may include an absorbent pad for receiving smaller volumes <not shown in fig>, where the pad may be composed of materials such as cellulose, nitrocellulose, or synthetic polymers that provide controlled sample absorption and release characteristics. In some aspects, the sample-introduction region 302 may include a direct application zone <not shown in fig> where the biological sample may be applied directly to an exposed portion of the primary fluidic pathway or to a specialized receiving surface. The sample-introduction region 302 may also include a dipping region configured to facilitate immersion of the test device 300 into a sample container, where the dipping region may be positioned and shaped to optimize sample uptake. Aspects of the present disclosure are intended to include or otherwise cover any type of material, without deviating from the scope of the present disclosure.

In some aspects, the sample-introduction region 302 may be located at different positions on the test device 300 depending on the intended use and flow configuration. In some aspects, the sample-introduction region 302 may be located at the top end of the primary fluidic pathway, where the biological sample may flow downward through the device 300 under the influence of gravity and capillary forces. The sample-introduction region 302 may be located at a bottom end of the primary fluidic pathway, where the biological sample may flow upward through capillary action or other transport mechanisms. In some aspects, the sample-introduction region 302 may be located at any other end or position along the primary fluidic pathway, where the specific positioning may be selected to optimize sample transport and distribution characteristics for a particular application. The positioning of the sample-introduction region described above is provided as an example only. The region may be placed at either end or at any suitable position along the primary fluidic pathway, depending on design requirements, intended use, and flow configuration. Aspects of the present disclosure are intended to include or otherwise cover any configuration of sample-introduction region, without deviating from the scope of the present disclosure.

At step 202, the application of the biological sample may result in obtaining a flowing sample along the primary fluidic pathway of the test device 300. The obtained flowing biological sample may be transported through the primary fluidic pathway by capillary action, where the dimensions and surface properties of the primary fluidic pathway may be configured to promote controlled sample flow. In some aspects, the obtained flowing sample may be facilitated by wicking materials or porous substrates that form the primary fluidic pathway and provide a continuous flow path for sample movement. In some aspects, the primary fluidic pathway may have a defined width, depth, and length that may be selected to provide appropriate flow rates and sample distribution characteristics. In some aspects, the primary fluidic pathway may extend in a generally straight direction, while in other aspects the pathway may include curves, bends, or other geometric features that may influence flow patterns.

In an embodiment, the primary fluidic pathway may have various configurations depending on the intended application and device design. In some aspects, the primary fluidic pathway may be formed as a channel or groove in a substrate material, where the channel may be defined by raised walls or barriers that contain the obtained flowing sample. The primary fluidic pathway may be formed by porous or fibrous materials that provide a continuous flow path through capillary action. In some aspects, the primary fluidic pathway may include multiple layers or components that work together to provide controlled sample transport, where different layers may have different porosity, surface chemistry, or flow characteristics. Aspects of the present disclosure are intended to include or otherwise cover any type of configuration of the primary fluidic pathway, without deviating from the scope of the present disclosure.

The configuration of the primary fluidic pathway may direct the applied biological sample to travel in various directions depending on the specific device design and application requirements. The term “primary fluidic pathway extending in a first direction” refers to the orientation and alignment of the main flow channel through which the applied biological sample travels. The first direction establishes the primary flow axis of the test device 300 and determines the overall flow pattern through the device. In some aspects, the configuration of the primary fluidic pathway may direct the applied biological sample to travel vertically along the primary fluidic pathway, where the first direction is vertical. Vertical flow configurations may provide controlled sample transport that may utilize gravitational forces in combination with capillary action to achieve consistent flow rates and sample distribution. The vertical sample transport may be accomplished through a primary fluidic pathway that extends in a generally downward direction from the sample-introduction region 302, where the pathway may be oriented substantially perpendicular to a horizontal plane. The vertical configuration may allow biological sample to flow downward under the influence of gravity while capillary forces within the pathway materials may provide controlled flow characteristics. When the primary fluidic pathway extends vertically, the first direction is the vertical direction from top to bottom of the device 300.

In some aspects, the configuration of the primary fluidic pathway may direct the applied biological sample to travel substantially perpendicular along the primary fluidic pathway, where the first direction is perpendicular to a reference plane. The substantially perpendicular travel may refer to sample flow that occurs at approximately 90 degrees relative to a reference plane or surface, where the perpendicular orientation may provide controlled flow characteristics and enable compact device designs. The substantially perpendicular configuration may be achieved through pathway geometries that orient the flow direction perpendicular to the device substrate or perpendicular to the sample-introduction region 302. When the primary fluidic pathway extends substantially perpendicular, the first direction is perpendicular to the plane of the test device 300 substrate.

In some aspects, the configuration of the primary fluidic pathway may direct the applied biological sample to travel angularly along the primary fluidic pathway, where the first direction is at an oblique angle. The angular travel may refer to sample flow that occurs at an angle other than vertical or horizontal, where the angle may be selected based on device design constraints, flow rate requirements, or detection zone positioning. The angular configuration may include flow at angles ranging from approximately 15 degrees to approximately 75 degrees relative to a horizontal plane, where the specific angle may be optimized for particular applications. The angular flow may provide advantages for certain device formats or may enable integration with specific sample collection or detection systems. When the primary fluidic pathway extends angularly, the first direction is the angular direction at the specified oblique angle.

Various embodiments of flow direction configurations may be employed depending on the specific device design and application requirements. In some aspects, the primary fluidic pathway may be formed as a vertical channel or groove that extends downward through a substrate material, where the channel walls may guide sample flow in the vertical direction. The vertical pathway may be formed by vertically oriented porous materials or fibrous substrates that may provide a continuous flow path through wicking action. In some aspects, the perpendicular configuration may include pathways that extend perpendicular to a device backing or support structure, enabling sample flow through the thickness of the device rather than along its length. The angular configuration may include pathways that extend at oblique angles, where the angled orientation may be achieved through shaped channels, angled substrates, or layered materials with directional flow properties. Aspects of the present disclosure are intended to include or otherwise cover any type of flow direction configuration, without deviating from the scope of the present disclosure.

In some aspects, flow direction may provide several advantages for the biological sample flow control and device performance. In some aspects, vertical flow may provide consistent flow rates that may be less dependent on device orientation or positioning during use. Vertical transport may facilitate controlled sample distribution to multiple detection zones by providing a central flow axis from which lateral branches may diverge symmetrically. In some aspects, perpendicular flow may allow for compact device designs where the one or more detection pads 304A-N may be arranged in a linear array along the perpendicular flow path, enabling sequential or simultaneous analyte quantification at multiple detection zones. Angular flow may provide flexibility in device design and may enable integration with various biological sample collection systems or detection platforms.

The method 200 may continue with enabling step 204 where bifurcation of the obtained flowing biological sample at one or more bifurcation nodes 306 (A-N) disposed along the primary fluidic pathway into at least two laterally directed fluid branches to flow towards one or more detection pads 304A-N arranged in an array along the primary fluidic pathway. The enabling step 204 may involve providing structural features and flow conditions that promote controlled sample division at the one or more bifurcation nodes 306 (A-N). In some aspects, the enabling step 204 may be accomplished through geometric features of the bifurcation nodes that direct portions of the obtained flowing sample into laterally extending channels or pathways. The one or more bifurcation nodes 306 (A-N) may include flow-diverting structures that may promote controlled sample division, where the structures may be shaped or positioned to achieve desired flow splitting ratios.

The enabling step 204 may occur when the obtained flowing sample encounters a bifurcation node that may be configured to split the sample flow into multiple streams. In some aspects, the bifurcation may occur through geometric features of the bifurcation node that direct portions of the sample into laterally extending channels or pathways. The one or more bifurcation nodes 306 (A-N) may include flow-diverting structures that may promote controlled sample division, where the structures may be shaped or positioned to achieve desired flow splitting ratios.

The one or more bifurcation nodes 306 (A-N) may have various structural configurations that may influence how sample division occurs during the enabling step 204. In some aspects, a bifurcation node may include a branching point where the primary fluidic pathway splits into two or more lateral branches, where the branching may be symmetrical or asymmetrical depending on the desired flow distribution. The bifurcation node may include flow-directing elements such as wedges, barriers, or channels that may guide sample flow into the lateral branches. In some aspects, the bifurcation node may include porous regions or materials with different flow characteristics that may promote sample division through differential capillary action or wicking rates. Aspects of the present disclosure are intended to include or otherwise cover any type of structural configurations, without deviating from the scope of the present disclosure.

The bifurcation of the obtained flowing sample at each bifurcation node (Hereinafter, the phrase “each bifurcation node” shall be understood to mean “each bifurcation node of the one or more bifurcation nodes 306 (A-N)”) of the one or more bifurcation nodes 306 (A-N) during the enabling step 204 may include dividing the sample into first and second lateral branches located on opposite sides of the primary fluidic pathway. The first and second laterally extending branches positioned on opposite sides of the primary fluidic pathway refers to a symmetric branching configuration where the first laterally extending branch extends from the bifurcation node toward one side of the primary pathway (such as the left side when viewing the device from a standard orientation) and the second laterally extending branch extends from the same bifurcation node toward the opposite side of the primary pathway (such as the right side). The opposite positioning creates a balanced, mirror-image flow pattern where sample is divided equally between the two sides of the primary pathway. The division into opposing lateral branches may provide symmetric sample distribution that may ensure equal sample delivery to detection zones positioned on either side of the primary pathway. In some aspects, the opposing configuration may create balanced flow patterns that may minimize flow disturbances and maintain consistent sample transport characteristics, which may be particularly important for accurate quantification where consistent sample volumes and flow rates at each detection pad may ensure reproducible signal generation.

The biological sample may split symmetrically at the one or more bifurcation nodes 306 (A-N) during the enabling step 204 through various mechanisms and configurations. In some aspects, symmetric splitting may be achieved through bifurcation node geometries that may present equal flow resistance to both lateral branches, where the flow resistance may be controlled through channel dimensions, surface properties, or material characteristics. The symmetric splitting may be facilitated by flow-diverting structures that may be positioned centrally within the primary pathway and may direct equal portions of sample flow toward opposing lateral branches. In some aspects, the splitting ratio may be controlled through differential channel dimensions or surface treatments that may create preferential flow distribution between the lateral branches. Different splitting ratios and configurations may be used during the enabling step 204 based on detection needs and device design. The split can be approximately equal, with each branch receiving similar sample volume or flow rate, or unequal, where one branch receives a larger portion for applications requiring varied volumes. In some cases, the ratio may be adjustable using flow-resistance elements or controllable diverting structures.

Each bifurcation node of the one or more bifurcation nodes 306 (A-N) may include a flow-diverting structure that promotes sample splitting during the enabling step 204. These structures, positioned at or near bifurcation points, use geometric features to create controlled flow patterns and pressure differentials for systematic division. Examples include wedge-shaped elements with apex angles of about 30°-90° for flow separation, and barrier elements partially obstructing the pathway (25%-75% of cross-sectional area) to redirect flow into lateral branches. Different geometries enable varied splitting ratios and flow control mechanisms. Aspects of the present disclosure are intended to include or otherwise cover any type of flow diverting structures, without deviating from the scope of the present disclosure.

The laterally directed fluid branches may extend from the one or more bifurcation nodes 306 (A-N) in directions transverse to the primary fluidic pathway. In some aspects, the laterally directed fluid branches may extend outward from the primary pathway at angles ranging from approximately 30 degrees to approximately 90 degrees relative to the primary flow direction. The lateral branches may then curve or redirect inward toward the one or more detection pads 304 (A-N), creating an outward-then-inward flow pattern that may provide controlled sample delivery to centrally positioned detection pads.

The configuration of the one or more bifurcation nodes 306 (A-N) and the laterally directed fluid branches may direct the obtained flowing sample to travel laterally outward from the primary fluidic pathway and subsequently laterally inward toward the one or more detection pads 304 (A-N). This outward-then-inward flow pattern may provide controlled biological sample delivery that may ensure uniform sample distribution to detection zones while maintaining flow continuity. In some aspects, the outward flow may occur when sample encounters a bifurcation node that may direct portions of the sample into lateral branches extending away from the primary pathway. The subsequent inward flow may occur as the lateral branches may curve or redirect sample flow toward the one or more detection pads 304 (A-N) positioned between opposing lateral branches.

The obtained flowing sample may progress along the primary fluidic pathway in a first direction and subsequently progress along the laterally directed fluid branches in a second direction transverse to the first direction. The first direction and second direction establish the two primary flow axes within the test device 300. The first direction corresponds to the primary flow axis along which the obtained flowing sample travels through the primary fluidic pathway, where the first direction may be vertical (downward from top to bottom), horizontal (laterally from one side to another), perpendicular (at 90 degrees to a reference plane), or angular (at an oblique angle). The second direction corresponds to the lateral flow axis along which the obtained flowing sample travels through the laterally directed fluid branches after bifurcation, where the second direction is transverse to the first direction. The term “transverse” means that the second direction is oriented at an angle relative to the first direction, typically at or near 90 degrees (perpendicular), though the angle may range from approximately 45 degrees to approximately 135 degrees. For example, when the first direction is vertical (downward), the second direction may be horizontal (outward to the sides), creating a perpendicular relationship between the two flow axes. When the first direction is horizontal, the second direction may be vertical or at an oblique angle. The transverse relationship between the first and second directions enables the bifurcated flow architecture where sample flows along the primary pathway in the first direction, then divides and flows laterally in the second direction toward the detection pads 304 (A-N). The first direction may correspond to the primary flow axis of the device 300, while the second direction may provide lateral sample distribution to multiple detection zones. In some aspects, the first direction may be vertical, horizontal, or at any other angle depending on the device orientation and intended use. The second direction may be perpendicular to the first direction, or may be at any other transverse angle that may provide effective sample distribution.

At step 206, the method 200 may include facilitating 206 the obtained flowing sample to pass through at least two successive bifurcation levels prior to reaching the one or more detection pads 304A-N. The facilitating step 206 may involve providing multiple tiers or stages of bifurcation nodes arranged sequentially along the primary fluidic pathway, where each bifurcation level may divide the obtained flowing sample into additional lateral branches. In some aspects, the facilitating 206 may enable systematic sample distribution to increasing numbers of the one or more detection pads 304 (A-N) through successive branching generations.

The facilitating step 206 may enable the obtained flowing sample to pass through successive bifurcation levels in a controlled manner that maintains consistent flow rates and sample volumes at each detection pad 304 (A-N). In some aspects, the successive bifurcation levels may be spaced at regular intervals along the primary fluidic pathway to provide uniform sample distribution timing.

The method 200 may further include allowing 208 the obtained flowing sample in at least two laterally directed fluid branches to contact the one or more detection pads 304A-N, each detection pad of the one or more detection pads 304 (A-N) including one or more biochemical agents configured to generate a signal. The allowing step 208 may involve enabling the obtained flowing sample that has been divided and directed through the lateral branches to reach and interact with the one or more detection pads 304 (A-N) where analyte detection and quantification occur.

The allowing step 208 may enable each detection pad of the one or more detection pads 304A-N (Hereinafter, the phrase “each detection pad” shall be understood to mean “each detection pad of the one or more detection pads 304A-N”) to receive obtained flowing sample from two laterally opposed branches. The dual-feed approach may ensure that the one or more detection pads 304 (A-N) receive adequate sample volume while providing improved sample mixing and distribution across the detection zone. In some aspects, the opposed lateral branches may deliver sample to the one or more detection pads 304A-N from opposite sides during the allowing step 208, where the converging flows may create uniform sample coverage and enhanced analyte-biochemical agent interactions.

The dual-feed approach during the allowing step 208 may work through various flow convergence mechanisms. In some aspects, the two opposed lateral branches may deliver sample to a detection pad simultaneously, where the converging flows may meet at or near the detection pad and may create mixing that may enhance analyte distribution and interaction kinetics. The opposed branches may deliver sample sequentially, where one branch may supply sample before the other, which may provide controlled sample application and processing. In some aspects, the dual-feed configuration may provide redundancy that may ensure sample delivery even if one lateral branch experiences flow restrictions or blockages.

The one or more detection pads 304 (A-N) may be arranged in an array along the primary fluidic pathway. In some aspects, the array may include a spaced tiered arrangement where the one or more detection pads 304A-N may be positioned at different vertical or horizontal levels along the primary fluidic pathway, where each detection pad may be separated from adjacent detection pads by a defined spacing. The spaced tiered arrangement may enable sequential or simultaneous sample delivery to multiple detection pads while maintaining controlled flow characteristics and preventing cross-contamination between detection zones.

Each detection pad of the one or more detection pads 304 (A-N) may include one or more biochemical agents configured to generate a signal during the allowing step 208. The one or more biochemical agents may be immobilized on or within the one or more detection pads 304 (A-N) to provide stable interaction sites for the one or more analytes present in the biological sample. In some aspects, the one or more biochemical agents may be selected based on the specific analytes to be detected and may provide high-affinity interactions that enable sensitive and specific analyte detection and quantification.

The one or more biochemical agents may include various types of binding molecules or detection reagents selected based on the target analytes and detection requirements. In some aspects, the one or more biochemical agents may include antibodies, antibody fragments, or engineered binding proteins that may provide specific recognition of the one or more analytes. The one or more biochemical agents may include nucleic acid probes, aptamers, or other oligonucleotide-based capture agents that may bind to specific target sequences or structures. In some aspects, the one or more biochemical agents may include enzymes, receptors, or other proteins that may interact specifically with the one or more analytes through binding, catalytic, or other molecular recognition mechanisms. The one or more biochemical agents may be selected from a group including, but not limited to, enzyme labels such as horseradish peroxidase (HRP), alkaline phosphatase (AP), glucose oxidase, or β-galactosidase that catalyze colorimetric or chemiluminescent reactions. In some aspects, the one or more biochemical agents may include fluorescent labels including fluorescein isothiocyanate (FITC), rhodamine derivatives, Alexa Fluor dyes, or cyanine dyes for optical detection. The one or more biochemical agents may include quantum dots providing enhanced photostability and narrow emission spectra. In some aspects, the one or more biochemical agents may include luminescent nanoparticles such as europium chelates or terbium complexes for time-resolved fluorescence detection. Aspects of the present disclosure are intended to include or otherwise cover any type of biochemical agents, without deviating from the scope of the present disclosure.

The allowing step 208 may enable the obtained flowing sample to interact with the one or more biochemical agents present at the one or more detection pads 304 (A-N). The interaction may involve specific molecular binding between target analytes in the sample and capture agents immobilized on the one or more detection pads 304 (A-N). In some aspects, the interaction may occur through antigen-antibody interactions, where antibodies specific to target analytes may be immobilized on the one or more detection pads 304 (A-N) and may capture corresponding antigens present in the sample. The interaction may involve nucleic acid hybridization, where complementary DNA or RNA sequences may bind to target nucleic acids in the sample. In some aspects, the interaction may involve enzymatic reactions where enzymes immobilized on the one or more detection pads 304 (A-N) may catalyze reactions with substrates present in the sample, generating detectable products.

The method 200 may include quantifying 210 biological sample by measuring the signal generated at the one or more detection pads 304 (A-N) to obtain an improved quantification output. The quantifying step 210 may involve measuring signal characteristics such as intensity, color development, fluorescence emission, or other quantifiable parameters and correlating these measurements with analyte concentrations or levels through calibration relationships. In some aspects, the quantifying 210 may be accomplished through comparison of measured signals with calibration standards or reference materials that may provide known analyte concentrations and corresponding signal responses.

The signal generated at the one or more detection pads 304 (A-N) during the quantifying step 210 may represent and indicate the concentration or level of the one or more analytes present in the biological sample. In some aspects, the signal intensity, color development, or other measurable characteristics may correlate with analyte concentration, allowing for quantitative analysis of the sample. The signal may provide qualitative information about analyte presence or absence, where signal detection above a threshold level may indicate positive analyte detection.

The signal may be colorimetric, fluorescent, chemiluminescent, radiometric, or electrochemical depending on the detection system and reagents employed. In some aspects, colorimetric signals may be generated through enzyme-substrate reactions that produce colored products, where the color intensity may correlate with analyte concentration. Fluorescent signals may be generated through fluorophore-labeled reagents that may emit light at specific wavelengths when excited by appropriate light sources. In some aspects, chemiluminescent signals may be produced through chemical reactions that generate light emission without external excitation. Radiometric signals may be generated through radioactive labels that may be detected using appropriate radiation detection equipment. Electrochemical signals may be generated through redox reactions or other electrochemical processes that may produce measurable electrical signals such as current, voltage, or impedance changes.

The signal intensity may correlate with concentration through various quantitative relationships during the quantifying step 210. The correlation may be non-linear, where the relationship between signal intensity and concentration may follow logarithmic, exponential, or other mathematical functions that may be characterized through calibration experiments. In some aspects, the correlation may include threshold effects where signal detection may occur only above minimum analyte concentrations, or saturation effects where signal intensity may plateau at high analyte concentrations.

The quantifying step 210 may be performed using various measurement techniques and instruments. In some aspects, the quantifying 210 may involve visual comparison of signal intensity or color development with reference color charts or calibration scales printed on or adjacent to the test device 300. In some aspects, quantifying 210 may involve optical measurement using dedicated readers, spectrophotometers, fluorescence detectors, or other analytical instruments that may quantify signal characteristics. In some aspects, the quantifying 210 may involve image capture using smartphone cameras or optical scanners, where captured images may be analyzed using image processing algorithms to extract quantitative signal information and calculate analyte concentrations.

In some aspects, the quantifying step 210 may be performed using a handheld imaging sensor configured to detect colorimetric, fluorescent, or chemiluminescent signals from the one or more detection pads 304A-N. The handheld imaging sensor may include optical components such as light-emitting diodes (LEDs) or laser diodes for excitation, optical filters for wavelength selection and background rejection, photodetectors such as photodiodes, photomultiplier tubes, or charge-coupled device (CCD) sensors for signal capture, and microprocessors with embedded algorithms for signal analysis, background subtraction, and concentration calculation. Aspects of the present disclosure are intended to include or otherwise cover any type of imaging sensor, without deviating from the scope of the present disclosure.

As used herein, the term “improved quantification output” refers to enhanced accuracy, precision, and reliability during the quantifying step 210, achieved through uniform sample distribution via a bifurcated dual-feed pathway, reduced signal variability across detection pads 304A-N, and the ability to measure multiple pads for statistical averaging, minimizing errors and edge effects.

The improved quantification output obtained during the quantifying step 210 may provide enhanced accuracy, precision, and reliability compared to traditional single-direction flow dipsticks. In some aspects, the improved quantification output may result from the uniform sample distribution provided by the bifurcated fluidic pathway architecture, where the dual-feed configuration ensures that all immobilized biochemical agents receive equal exposure to analytes. The improved quantification output may result from reduced signal variability across the one or more detection pads 304A-N, where the converging flows from opposed lateral branches eliminate edge effects and incomplete reagent wetting. In some aspects, the improved quantification output may result from the ability to measure at one or more detection pads 304 (A-N) simultaneously or sequentially, where statistical averaging of multiple measurements may reduce measurement errors and improve quantification precision.

In an embodiment, method 200 may enable pregnancy testing in urine samples using a bifurcated fluidic pathway. The method includes applying 202 the sample to the introduction region 302, where it flows along the primary pathway and splits at a bifurcation node into two lateral branches. These branches deliver the sample to a centrally positioned detection pad 304A during the allowing step 208, ensuring uniform distribution through converging flows. The pad 304A may contain anti-hCG antibodies to interact with hCG, with the dual-feed design improving analyte exposure and signal strength compared to single-feed systems. This configuration enables detection of hCG at concentrations as low as 10-25 mIU/mL for early pregnancy detection. Quantifying 210 may involve measuring colorimetric intensity visually against reference standards or via optical analysis using a smartphone-based system.

In another embodiment, method 200 may enable fertility tracking by detecting multiple reproductive hormones using successive bifurcation levels. A urine sample applied to the introduction region 302 flows through at least two bifurcation stages before reaching detection pads 304 (A-N). At the first level, the sample splits into two lateral branches; at the second, each branch divides again, directing flow to multiple pads arranged along the primary pathway. Pads may include anti-LH, anti-E3G, and anti-PdG antibodies for detecting luteinizing hormone, estradiol glucuronide, and pregnanediol glucuronide, respectively. Each pad receives sample from opposed branches, ensuring uniform volume and analyte exposure. This architecture eliminates multiple dipping steps and enhances signal generation for all analytes simultaneously. Quantification 210 may involve measuring signal intensities via optical scanners or smartphone-based systems to determine hormone levels for identifying fertile windows and tracking cycle phases.

In another embodiment, method 200 may enable comprehensive urinalysis by detecting six or more analytes using multiple bifurcation levels that systematically distribute sample to detection pads 304 (A-N). A urine sample applied to the introduction region 302 flows through sequential bifurcation nodes, each dividing the sample into lateral branches that supply pads from opposing sides. This architecture overcomes a key limitation of conventional dipsticks, where downstream pads often receive insufficient or depleted sample, by ensuring each pad receives fresh sample directly from the primary pathway. Pads may detect proteins, blood, glucose, ketones, pH, and specific gravity, with dual-feed delivery promoting uniform distribution and complete reagent activation. Quantification 210 may involve measuring signal intensities at each pad using an optical scanner for accurate multi-parameter analysis.

In another embodiment, method 200 may be adapted for point-of-care or home testing, offering improved reliability and ease of use over traditional dipsticks. A biological sample may be applied 202 by dipping or direct application, with the bifurcated pathway accommodating variations in technique. Unlike conventional strips where incomplete immersion can cause uneven sample distribution, the primary pathway and bifurcation nodes systematically deliver sample to all detection pads 304 (A-N) during enabling step 204. Dual-feed delivery from opposed branches ensures adequate sample even with limited volume, promoting uniform color development. Results may be visually interpreted or quantified 210 using smartphone imaging and app-based analysis, which can extract signal intensities and display results as numerical values, trends, or color-coded indicators.

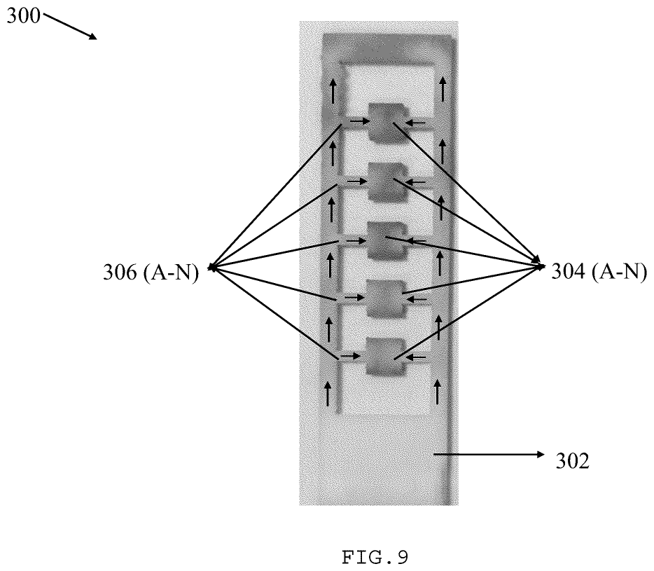

Referring to FIG. 9, the test device 300 for detecting one or more analytes in a biological sample may provide a structured platform that enables systematic execution of the detection method 200 through integrated fluidic components and controlled sample processing pathways. The test device 300 may facilitate controlled sample distribution and analyte quantification through specialized structural features that guide sample flow and enable specific interaction reactions at designated detection zones. In some aspects, the test device 300 may be implemented as a lateral flow assay device that provides convenient sample application and processing capabilities suitable for various analytical applications.

The test device 300 may include the sample-introduction region 302 configured to receive the biological sample and facilitate the applying step 202 of the method 200. The sample-introduction region 302 may provide a controlled interface between the sample and the fluidic network of the device 300, where the region may be designed to accommodate various sample types and application methods. In some aspects, the sample-introduction region 302 may be configured to receive samples through direct contact, immersion, or dispensing methods depending on the specific application requirements and sample handling preferences.

Various embodiments of sample-introduction configurations may be employed to accommodate different sample volumes and application methods. In some aspects, the sample-introduction region 302 may include a well structure for receiving and holding sample volumes, where the well may provide a defined reservoir that may retain sample before controlled release into the primary fluidic pathway. The well structure may have a capacity ranging from approximately 10 microliters to approximately 1000 microliters, where the specific volume may be selected based on detection sensitivity requirements and sample availability. The well may have various geometric configurations such as circular, rectangular, or irregular shapes that may optimize sample retention and flow initiation characteristics. Aspects of the present disclosure are intended to include or otherwise cover any type of sample-introduction configuration, without deviating from the scope of the present disclosure.

In some aspects, the sample-introduction region 302 may include an absorbent pad made of porous materials such as cellulose, cotton, glass fiber, or polymer membranes for wicking and controlled release. Alternatively, the region may include a dipping section for immersion-based application, featuring hydrophilic surfaces to promote uptake and to ensure adequate contact during immersion.

The test device 300 may include a primary fluidic pathway extending in a first direction from the sample-introduction region 302 and facilitating sample transport through controlled sample movement mechanisms. The term “primary fluidic pathway extending in a first direction” refers to the orientation and spatial arrangement of the main flow channel through which the received biological sample travels after application to the sample-introduction region 302. The first direction establishes the primary flow axis of the test device 300 and determines the overall flow pattern and device orientation. The primary fluidic pathway may provide a main conduit for sample transport that may connect the sample-introduction region 302 to the bifurcation network and detection zones. In some aspects, the primary fluidic pathway may be configured to provide controlled flow rates and consistent sample transport characteristics that may ensure reliable sample delivery to downstream components.

Various embodiments of pathway structures may enable sample transport through different physical mechanisms and material configurations. In some aspects, the primary fluidic pathway may be formed as a channel or groove in a substrate material, where the channel may be defined by raised walls or barriers that may contain and guide the flowing sample. The channel configuration may have controlled dimensions including width, depth, and length that may be selected to provide appropriate capillary forces and flow resistance for specific sample types and flow rate requirements. The channel walls may have heights ranging from approximately 0.1 millimeters to approximately 1 millimeter, where the wall height may be selected to prevent sample overflow while maintaining open access for optical measurements.

The primary fluidic pathway may be formed from porous or fibrous materials such as nitrocellulose, cellulose, glass fiber, or polymer membranes, providing capillary-driven flow without external pressure. These materials may have pore sizes with smaller pores enhancing capillary forces and larger pores enabling faster flow. Surface treatments like hydrophilic coatings or surfactants may optimize wetting and flow. Aspects of the present disclosure are intended to include or otherwise cover any type of materials, without deviating from the scope of the present disclosure.

The test device 300 may include a substrate or backing layer that provides structural support for the fluidic pathway and components. The substrate may be formed from rigid or semi-rigid materials such as plastic, cardboard, or laminated composites to ensure rigidity and compactness. Adhesive regions may secure the primary pathway, lateral branches, and detection pads 304A-N. In some aspects, a protective cover or laminate may overlay the pathway and pads to provide environmental protection while allowing optical access. The cover may be transparent or translucent, formed from materials such as clear plastic films or polymer laminates, and may include viewing windows aligned with detection pads for unobstructed signal measurement.

In some aspects, the configuration of the primary fluidic pathway may direct the received biological sample to travel in various directions depending on the specific device design and application requirements. In some aspects, the configuration of the primary fluidic pathway may direct the received biological sample to travel vertically along the primary fluidic pathway, where the first direction is vertical. The vertical pathway configuration may provide controlled sample transport that may utilize gravitational forces in combination with capillary action to achieve consistent flow rates and sample distribution. The vertical orientation may be achieved by positioning the sample-introduction region 302 at the top of the device 300 and orienting the primary fluidic pathway to extend downward, where the pathway may be aligned substantially perpendicular to a horizontal plane when the device 300 is held in a vertical orientation. When the primary fluidic pathway extends vertically, the first direction is the vertical direction from top to bottom of the device 300, and the received biological sample flows downward along this vertical axis.

In some aspects, the configuration of the primary fluidic pathway may direct the received biological sample to travel substantially perpendicular to a reference plane, where the first direction is perpendicular. The substantially perpendicular configuration may be achieved through pathway geometries that orient the flow direction perpendicular to the device substrate or perpendicular to the sample-introduction region 302. The perpendicular orientation may enable compact device designs where the detection pads 304 (A-N) may be arranged in a linear array along the perpendicular flow axis. When the primary fluidic pathway extends substantially perpendicular, the first direction is perpendicular to the plane of the device substrate, and the received biological sample flows along this perpendicular axis.

In some aspects, the configuration of the primary fluidic pathway may direct the received biological sample to travel angularly along the primary fluidic pathway, where the first direction is at an oblique angle. The angular configuration may include flow at angles ranging from approximately 15 degrees to approximately 75 degrees relative to a horizontal plane, where the specific angle may be optimized for particular applications. The angular orientation may be achieved through angled substrate positioning, angled pathway construction, or device designs that incorporate angled flow sections. When the primary fluidic pathway extends angularly, the first direction is the angular direction at the specified oblique angle, and the received biological sample flows along this angled axis.

The test device 300 may include one or more bifurcation nodes 306 (A-N) disposed along the primary fluidic pathway, where each bifurcation node may be configured to divide a portion of the received biological sample flowing along the primary fluidic pathway into first and second laterally extending branches positioned on opposite sides of the primary fluidic pathway. The one or more bifurcation nodes 306 (A-N) may enable the enabling step 204 of the method 200 by providing controlled sample splitting that may distribute sample aliquots to multiple detection zones while maintaining flow continuity in the primary pathway.

Each bifurcation nodes may be positioned along the primary fluidic pathway at locations corresponding to detection pad positions, spaced to ensure systematic sample distribution. Each bifurcation node may be placed upstream of its associated pad to divide the flowing sample before delivery. Each bifurcation node may incorporate branching geometries that split the primary flow into lateral branches, directing portions of the sample toward opposite sides of the pathway.

In some aspects, flow-diverting structures such as wedges, barriers, or shaped channel features may guide sample into the branches while maintaining continuity in the main pathway. A wedge-shaped element may be centrally positioned at the bifurcation point to create flow separation toward lateral openings, with its geometry optimized for effective diversion without blocking the main flow. Alternatively, a barrier element may partially obstruct the primary pathway to redirect flow toward lateral branches. The barrier may be formed from non-wicking material, a hydrophobic region, or a physical obstruction that limits central flow and increases local velocity, promoting diversion into lateral channels. These structures enable controlled splitting ratios and uniform sample delivery across all one or more detection pads (304 A-N).

In some aspects, the bifurcation node may include lateral openings or channels that extend from the primary pathway toward the one or more detection pads 304 (A-N). In some aspects, the lateral openings may be formed by gaps, cuts, or shaped regions in the pathway material that allow sample to flow laterally away from the primary axis.

The first and second laterally extending branches positioned on opposite sides of the primary fluidic pathway refers to a symmetric branching configuration where the branches extend from a bifurcation node in opposing directions. The first laterally extending branch extends from the bifurcation node toward one side of the primary fluidic pathway, such as the left side when viewing the device 300 from a standard front-facing orientation. The second laterally extending branch extends from the same bifurcation node toward the opposite side of the primary fluidic pathway, such as the right side. The opposite positioning creates a balanced, mirror-image flow pattern where the biological sample received is divided equally between the two sides of the primary pathway. The first and second branches are positioned symmetrically relative to the centerline or central axis of the primary fluidic pathway, ensuring that each branch receives substantially equal sample aliquots. The opposing branch arrangement may ensure that sample flow may be divided approximately equally between the two branches at each bifurcation node, where the equal division may provide consistent sample delivery to the one or more detection pads 304 (A-N) supplied by the branch pairs. In some aspects, the opposing configuration may create flow patterns that minimize flow disturbances in the primary pathway while providing controlled lateral sample distribution.

Each laterally extending branch may be made from porous or fibrous materials similar to those of the primary fluidic pathway, enabling continuous capillary flow from the bifurcation node to the detection pad. These branches may use the same material as the primary pathway for uniform flow or different materials to achieve specific flow rates or processing functions.

Branches may include outward segments directing flow away from the primary pathway and inward segments redirecting flow toward detection pads. Outward segments may extend at angles of about 30°-90°. Transition regions between these segments may feature smooth curves with smaller radii minimize resistance, or angled bends changing direction by about 60°-120°.

The primary fluidic pathway may extend in a first direction, and each laterally extending branch may extend in a second direction transverse to the first direction. The first direction may correspond to the primary flow axis of the device 300, while the second direction may provide lateral sample distribution to multiple detection zones positioned away from the central flow axis. In some aspects, the transverse relationship between the first and second directions may enable compact device designs where multiple detection zones may be arranged efficiently within the available device footprint. The angle between the first and second directions may range from approximately 45 degrees to approximately 135 degrees, where angles closer to 90 degrees may provide the most efficient lateral distribution.

The test device 300 may include the one or more detection pads 304 (A-N) arranged in an array along the primary fluidic pathway, where each detection pad of the one or more detection pads 304 (A-N) may be fluidically connected to a respective pair of the laterally extending branches and enabling the sample to flow (i) along the primary fluidic pathway, and subsequently (ii) outward and inward toward the one or more detection pads 304 (A-N). The detection pad configuration may enable the allowing step 208 and quantifying step 210 of the method 200 by providing controlled sample delivery and specific analyte interaction zones where reactions may occur.

Each detection pad 304 (A-N) may be formed from porous, protein-binding membranes such as nitrocellulose, nylon, or PVDF, providing a substrate for immobilizing one or more biochemical agents like antibodies or enzymes. Aspects of the present disclosure are intended to include or otherwise cover any type of materials, without deviating from the scope of the present disclosure.

Each detection pad may be positioned between the pair of laterally extending branches supplying the sample. The positioning of the one or more detection pads 304 (A-N) between opposing branches may enable sample delivery from two converging flow streams that may approach the detection pad from opposite sides. In some aspects, the dual-feed arrangement may provide increased sample volume delivery compared to single-branch configurations while creating mixing effects that may enhance analyte distribution and interaction kinetics. The one or more detection pads 304 (A-N) may be positioned such that the inward-extending segments of the lateral branches contact opposite edges of each detection pad.

Each detection pad of the one or more detection pads 304A-N may receive the biological sample from two inwardly converging flows. The converging flow configuration may create turbulent mixing at detection pad surfaces that improves analyte distribution and increases the probability of analyte-biochemical agent interactions. In some aspects, the inward convergence may provide increased sample contact time and enhanced mass transfer that improves interaction kinetics and detection sensitivity. The converging flows may meet at or near the center of each detection pad, where the convergence point may be positioned to ensure uniform sample distribution across the entire pad surface.

Each detection pad of the one or more detection pads 304 (A-N) may be positioned along a central axis relative to the pair of laterally extending branches fluidically connected to the detection pad. The central axis positioning may ensure that the one or more detection pads 304 (A-N) receive balanced sample delivery from paired extending branches while maintaining consistent geometric relationships that optimize flow characteristics. In some aspects, the central positioning may create symmetric flow patterns that enhance mixing and improve detection performance through uniform sample coverage. The detection pads 304 (A-N) may be aligned along a vertical centerline of the device 300, where the centerline may coincide with the central axis of the primary fluidic pathway.

The array of detection pads 304 (A-N) may include a spaced tiered arrangement along the primary fluidic pathway. The spaced tiered arrangement may position one or more detection pads 304 (A-N) at regular intervals along the device length, where each detection pad may be separated from adjacent detection pads by a defined spacing. The spaced tiered arrangement may enable detection of multiple analytes or multiple concentrations of the same analyte through different detection pads with varying biochemical agent specificities or concentrations. The spaced tiered arrangement may facilitate optical scanning and automated signal measurement, where optical analysis systems may sequentially measure signals at each detection pad position along a defined scan path.

In some aspects, the test device 300 may include from two to ten detection pads 304 (A-N) arranged in the spaced tiered arrangement, where the specific number may be selected based on the number of analytes to be detected or the number of concentration measurements required. In some aspects, devices configured for single-analyte quantification may include three to eight detection pads that all detect the same analyte, while devices configured for multi-analyte detection may include two to ten detection pads where each pad detects a different analyte. Aspects of the present disclosure are intended to include or otherwise cover any number of detection pads, without deviating from the scope of the present disclosure.

Each detection pad of the one or more detection pads 304 (A-N) may include one or more biochemical agents configured to generate signals that enable quantification of an analyte level across the array of detection pads 304 (A-N). The one or more biochemical agents may be immobilized on pad surfaces or within pad materials, where the one or more biochemical agents may provide specific recognition and interaction with the one or more analytes present in the sample. The one or more biochemical agents may be selected from a group including, but not limited to, enzyme labels such as horseradish peroxidase (HRP), alkaline phosphatase (AP), glucose oxidase, or β-galactosidase that catalyze colorimetric or chemiluminescent reactions, fluorescent labels including fluorescein isothiocyanate (FITC), rhodamine derivatives, Alexa Fluor dyes, or cyanine dyes for optical detection, quantum dots providing enhanced photostability and narrow emission spectra, or luminescent nanoparticles such as europium chelates or terbium complexes for time-resolved fluorescence detection. Aspects of the present disclosure are intended to include or otherwise cover any type of biochemical agents, without deviating from the scope of the present disclosure.

The one or more biochemical agents may be immobilized on the detection pads 304A-N through various immobilization techniques. In some aspects, the one or more biochemical agents may be applied to the one or more detection pads 304 (A-N) through spotting, spraying, or printing techniques that deposit controlled amounts of reagent solutions onto specific pad locations. The biochemical agents may be applied in solution form and allowed to dry, where the drying process may result in passive adsorption of the agents onto the membrane surface. In some aspects, the biochemical agents may be covalently attached to the membrane surface through chemical crosslinking reactions using reagents such as glutaraldehyde, carbodiimides, or other bifunctional crosslinkers.

In some aspects, the one or more detection pads 304 (A-N) may include blocking agents or stabilizing reagents that may prevent non-specific binding and maintain biochemical agent activity during storage. In some aspects, the blocking agents may include proteins such as bovine serum albumin (BSA), casein, or milk proteins that may occupy non-specific binding sites on the membrane surface. The on or more detection pads 304 (A-N) may include stabilizing reagents such as sugars (trehalose, sucrose), polymers (polyvinylpyrrolidone, polyethylene glycol), or preservatives (sodium azide, ProClin) that may maintain biochemical agent activity and prevent microbial growth during storage. Aspects of the present disclosure are intended to include or otherwise cover any type of blocking agents or stabilizing reagents, without deviating from the scope of the present disclosure.

In some aspects, the test device 300 may further include a flow-termination region disposed downstream of the lowermost bifurcation node. The flow-termination region may provide a controlled endpoint for sample flow that prevents sample overflow while ensuring complete sample delivery to all detection zones. In some aspects, the flow-termination region may include absorbent materials or reservoir structures that capture excess sample and maintain controlled flow characteristics throughout the test device 300. The flow-termination region may be formed from highly absorbent materials such as cellulose pads, glass fiber pads, or synthetic absorbent polymers that may have high sample capacity.

Each pair of laterally extending branches may deliver substantially equal aliquots of the sample to a detection pad. The equal aliquot delivery may provide consistent sample volumes and concentrations at each detection pad, enabling reliable analyte interaction and signal generation. In some aspects, equal delivery may be achieved through symmetric branch geometries and flow characteristics that create balanced flow resistance and uniform sample splitting at the one or more bifurcation nodes 306 (A-N). The lateral branches may have identical dimensions, materials, and flow path lengths to ensure equal flow resistance and equal sample delivery rates.

The configuration of the primary fluidic pathway, the one or more bifurcation nodes 306 (A-N), and the laterally extending branches may direct the sample to flow along the primary fluidic pathway, and subsequently outward and inward toward the one or more detection pads 304A-N. This integrated flow architecture may enable the complete method 200 by providing systematic sample processing that ensures controlled sample delivery to all detection zones with consistent timing and volume characteristics.

In some aspects, the test device 300 may include a handle region or grip area that may facilitate user handling during sample application and result reading. In some aspects, the handle region may be positioned at an end of the device opposite the sample-introduction region 302, where the handle may provide a clean gripping surface that remains free from sample contact. The handle region may include textured surfaces, finger grips, or ergonomic features that improve handling comfort and control.

In operation, the device 300 draws a biological sample from the introduction region 302 into the primary fluidic pathway by capillary action. The sample flows in a first direction along the pathway (vertical, perpendicular, or angular depending on configuration) and encounters sequential bifurcation nodes. Each node divides the flow into two lateral branches extending outward in a second, transverse direction, then redirects inward toward detection pads 304A-N. Each pad receives converging flows from opposite branches for uniform distribution, ensuring consistent analyte interaction with immobilized agents. Signals generated at the pads—colorimetric, fluorescent, chemiluminescent, radiometric, or electrochemical, correlate with analyte concentration and are read visually or via optical systems. Excess samples are absorbed in the termination region, maintaining controlled flow and preventing overflow.

In another embodiment, the test device 300 enhances detection and quantification compared to single-direction dipsticks through its bifurcated fluidic pathway. One or more bifurcation nodes 306 (A-N) split the biological sample into lateral branches feeding one or more detection pads 304A-N from opposite sides, creating a dual-feed configuration for uniform distribution. This eliminates edge effects and incomplete wetting seen in available dipsticks, preventing signal gradients. Uniform flow improves sensitivity by ensuring equal analyte exposure, increases capture efficiency, and lowers detection limits. It also enhances quantification accuracy by producing consistent signals for reliable optical measurement, reduces assay time through faster pad wetting, and improves reproducibility by delivering equal aliquots to each detection pad. Symmetric flow patterns along the central axis maintain consistent performance across tests.

In another embodiment, the test device 300 may be configured for high-accuracy quantification using the uniform signal development provided by the bifurcated fluidic pathway. One or more detection pads 304A-N receive the biological sample from opposed lateral branches, where the dual-feed design ensures consistent signal formation. Reference features such as calibration patches, color standards, or fiducial marks may be positioned adjacent to the pads to enable optical systems to correct for lighting or camera variations. These features, applied at fixed positions on the substrate or cover layer, may include color patches with defined reflectance or fluorescent standards with known emission values. Uniform flow improves calibration accuracy by eliminating flow artifacts, ensuring signals reflect true analyte concentrations. Multiple pads detecting the same analyte may be supplied by respective bifurcation nodes for equal sample delivery, with quantification based on averaged readings to reduce error. The test device 300 supports optical analysis systems that capture pad images and extract signal intensities via image processing, where uniform distribution simplifies automated analysis. A tiered arrangement of pads along the primary pathway enables sequential scanning for individual calibration and quality checks.

In another embodiment, the test device 300 may operate effectively with reduced sample volumes through efficient distribution enabled by the bifurcated fluidic pathway. The sample-introduction region 302 may receive volumes of about 10-200 μL, which are split at bifurcation nodes and delivered to one or more detection pads 304A-N via opposed lateral branches. This dual-feed design ensures adequate sample delivery even with limited volume and eliminates waste. The primary pathway and branches may be dimensioned to maintain controlled flow rates of about 50-500 μL/min, providing sufficient contact time without stagnation or overflow. Porous materials may have pore sizes of about 5-15 μm to balance capillary forces and flow resistance. The architecture accommodates samples of varying viscosity while maintaining uniform delivery. A downstream flow-termination region absorbs excess sample, with capacity of about 100-1000 μL, ensuring controlled flow throughout the process.

In another embodiment, the test device 300 may enable quantitative detection of a single analyte using one or more detection pads 304A-N arranged in a spaced tiered layout along the primary fluidic pathway. Each detection pad includes the same biochemical agents and receives sample via opposed lateral branches from respective bifurcation nodes, ensuring equal sample delivery and uniform signal development. This configuration offers key advantages: fresh sample to each detection pad, consistent binding conditions, and improved accuracy through averaging multiple independent measurements. Optical analysis systems, including smartphone cameras or scanners, capture pad images for signal extraction and quantification. The bifurcated architecture ensures equal aliquots for direct comparison, prevents cross-contamination and supports independent optical measurement.

In another embodiment, the test device 300 may enable simultaneous detection and quantification of multiple analytes using two to ten detection pads 304A-N arranged in a spaced tiered layout along the primary fluidic pathway. Each detection pad includes biochemical agents specific to a different analyte and receives sample via opposed lateral branches from respective bifurcation nodes, ensuring equal delivery and uniform signal development. This configuration allows comprehensive analysis from a single sample, such as panels for proteins, metabolites, pH, or hormones. The bifurcated architecture eliminates analyte depletion seen in traditional dipsticks and ensures consistent activation across all pads. Pad spacing of about 8-20 mm prevents cross-contamination and supports independent optical measurement. Biochemical agents may include antibodies for proteins, enzymatic reagents for metabolites, and pH or ion-selective indicators for sample properties. The test device 300 supports diagnostic panels like urinalysis (proteins, glucose, ketones, pH, specific gravity) or fertility panels (LH, estrogen, progesterone metabolites). Optical analysis systems may sequentially scan all pads and generate a comprehensive analyte profile displayed as numerical values, graphs, or color-coded indicators.

In another embodiment, a system for analyte quantification may include the test device 300 and an external device configured for optical measurement, signal analysis, and conversion of optical signals into concentration values. In some aspects, the external device may be implemented as a portable reader, smartphone-based system, or dedicated optical reader. A portable reader may include optical components, processing electronics, and a slot for consistent positioning of the test device. In some aspects, a smartphone-based system may use an adapter for controlled alignment and lighting, with an app for image capture, signal processing, and calculation. A dedicated optical reader may include specialized optics and algorithms optimized for the test device format. Aspects of the present disclosure are intended to include or otherwise cover any type of external device, without deviating from the scope of the present disclosure.

The external device may detect colorimetric, fluorescent, or chemiluminescent signals using light sources for illumination or excitation and sensors such as CCD/CMOS cameras or photodiode arrays equipped with filters and lenses. Aspects of the present disclosure are intended to include or otherwise cover any type of light sources, without deviating from the scope of the present disclosure. Processing algorithms may perform background correction, apply calibration factors, and convert signals into analyte concentrations. Display interfaces may present results in numerical or graphical formats, while communication interfaces (Bluetooth, Wi-Fi, USB) enable data transfer to external systems for storage or analysis. Aspects of the present disclosure are intended to include or otherwise cover any type of communication interfaces, without deviating from the scope of the present disclosure. Aspects of the present disclosure are intended to include or otherwise cover any type of display interfaces, without deviating from the scope of the present disclosure.

In another embodiment, the test device 300 may be configured for pregnancy testing, including a sample-introduction region 302 at the top of a vertically oriented primary fluidic pathway. The pathway extends downward and includes a bifurcation node above a detection pad 304A, which splits the urine sample into first and second lateral branches that curve inward to converge at the pad. This dual-feed design ensures complete wetting, eliminates edge effects, and enhances hCG distribution. On or more detection pads 304 (A-N) may include anti-hCG antibodies immobilized on a nitrocellulose membrane. Colorimetric signals using gold nanoparticle conjugates may develop rapidly, providing uniform color for accurate visual interpretation and automated optical analysis. The device may support smartphone-based quantification via image capture and signal intensity measurement, delivering both qualitative and numerical results.

In another embodiment, the test device 300 may be configured for comprehensive fertility monitoring with three or more detection pads 304 (A-N) arranged in a spaced tiered layout along a vertically oriented primary fluidic pathway. Multiple bifurcation nodes positioned at intervals of about 12 mm supply respective pads via opposed lateral branches, ensuring fresh sample delivery, uniform distribution, and equal aliquots for consistent performance. Detection pads may include anti-LH antibodies (304A) for ovulation prediction, anti-E3G antibodies (304B) for estrogen metabolite monitoring, and anti-PdG antibodies (304C) for confirming ovulation. The test device 300 requires smaller volume of urine and supports optical scanners or smartphone-based analysis to quantify hormone levels and track fertility patterns over time.

In another embodiment, the test device 300 may function as a multi-parameter urinalysis panel with six to ten detection pads 304 (A-N) arranged in a spaced tiered layout along a vertically oriented primary fluidic pathway. This architecture ensures fresh sample delivery, uniform wetting, and equal aliquots, eliminating analyte depletion and cross-contamination common in traditional dipsticks. One or more detection pads (304 A-N) may include reagents for protein (304A), blood (304B), glucose (304C), ketones (304D), pH (304E), and specific gravity (304F), each about 5 mm×5 mm and formed from membranes suited to their chemistry. The device provides simultaneous analysis within ˜60 seconds and supports optical scanners for sequential signal measurement and quantification.

In another embodiment, the test device 300 may be designed for home use, where the bifurcated pathway architecture simplifies operation compared to traditional dipsticks. The sample-introduction region 302 may include a wide dipping area for easy immersion, drawing sample by capillary action without precise timing. Bifurcation nodes automatically distribute the sample to all detection pads 304 (A-N), eliminating the need for full immersion. Pads are visible through a result window in the cover layer, and dual-feed flow ensures uniform, intense color for easier interpretation. The device may include a handle with textured grip and a protective cap for storage, packaged in sealed foil with desiccant. Compatible smartphone apps may guide users with step-by-step instructions and analyze pad images for automated results. Uniform color development improves image-based accuracy. Results are available rapidly, and sample adequacy indicators confirm proper application.

A number of implementations have been described. Nevertheless, it will be understood that various modifications may be made without departing from the spirit and scope of the disclosure. Accordingly, other implementations are within the scope of the following claims.

### Advantages of the Invention

The present invention provides several significant advantages over traditional lateral flow assays and dipstick-based diagnostic devices:
1. Enhanced Quantification Accuracy: The bifurcated fluidic pathway architecture with dual-feed configuration to each detection pad 304 (A-N) provides improved quantification accuracy by ensuring uniform sample distribution across the entire detection pad surface. The converging flows from two opposed lateral branches eliminate edge effects and signal gradients that commonly occur in single-direction flow devices, thereby enabling more reliable optical measurements and precise analyte concentration determination during the quantifying step 210. The uniform signal development facilitates automated image analysis and reduces measurement errors caused by signal gradients or localized variations.
2. Improved Detection Sensitivity: The dual-feed approach during the allowing step 208 ensures that all immobilized biochemical agents on each detection pad 304 (A-N) receive equal exposure to analytes through symmetric sample delivery from opposite sides. This uniform analyte exposure increases the probability of analyte-biochemical agent interactions, thereby improving binding efficiency and lowering detection limits compared to traditional dipsticks where incomplete reagent wetting results in reduced sensitivity. Enhanced biological sample delivery through bifurcated pathways enables detection at lower analyte concentrations.
3. Reduced Sample Volume Requirements: The bifurcated pathway architecture enables efficient sample usage by delivering fresh sample directly from the primary pathway to each detection pad 304A-N through dedicated lateral branches during the enabling step 204. This systematic sample distribution eliminates sample waste that occurs in traditional dipsticks where sample must flow sequentially through multiple pads, thereby allowing the test device 300 to operate effectively with smaller sample volumes while maintaining adequate sample delivery to all detection zones. The controlled splitting during the enabling step 204 ensures that each detection pad receives adequate sample volume even when the total applied sample volume is limited.
4. Elimination of Analyte Depletion Effects: The sequential bifurcation architecture supplies each detection pad 304A-N directly from the primary fluidic pathway through dedicated lateral branches, thereby ensuring that all pads receive fresh sample with consistent analyte concentrations. This configuration overcomes a critical limitation of traditional multi-parameter dipsticks where downstream pads receive sample depleted of analytes by upstream pads, resulting in unreliable readings for parameters positioned at the end of the strip. Each detection pad receives sample directly from the primary pathway rather than receiving pre-depleted sample that has already contacted upstream pads.
5. Enhanced Reproducibility and Precision: The controlled sample splitting at the one or more bifurcation nodes 306 (A-N) during the enabling step 204 delivers substantially equal sample aliquots to each detection pad 304 (A-N), thereby reducing pad-to-pad variations in signal intensity. The symmetric flow patterns created by the dual-feed configuration provide consistent flow characteristics across multiple tests, thereby improving assay reproducibility and enabling more reliable quantitative measurements compared to traditional devices where flow variations significantly affect results. The bifurcated flow architecture ensures that all detection pads receive substantially equal sample aliquots, enabling direct comparison of signal intensities across pads without requiring normalization for sample volume variations.
6. Faster Assay Times: The bifurcated pathway architecture enables faster assay times by delivering sample more rapidly to detection zones through multiple parallel flow paths. The dual-feed configuration reduces the time required for complete pad wetting and analyte-biochemical agent interaction, thereby enabling faster color development and shorter time-to-result compared to traditional single-direction flow devices where the biological sample must travel longer distances to reach all detection pads. Enhanced biological sample delivery through bifurcated pathways enables faster assay times compared to traditional designs.
7. Reduced Susceptibility to Sample Application Variations: the Test Device 300 Demonstrates reduced susceptibility to variations in sample application technique compared to traditional dipsticks. The sample-introduction region 302 feeds into a single primary fluidic pathway that systematically distributes sample to all detection pads 304 (A-N) through the bifurcation nodes during the enabling step 204, regardless of variations in immersion depth or application angle. This design reduces result variability arising from inconsistent sample application and improves result reliability in point-of-care and home testing scenarios where sample application techniques may vary. The bifurcated pathway architecture ensures that all pads receive adequate sample delivery despite variations in the initial sample application method.

We claim:

1. A method for detecting one or more analytes in a biological sample, the method comprising:
a. applying the biological sample to a sample-introduction region of a test device to obtain a flowing sample along a primary fluidic pathway of the test device;
b. enabling bifurcation of the obtained flowing sample at one or more bifurcation nodes disposed along the primary fluidic pathway into at least two laterally directed fluid branches to flow towards one or more detection pads arranged in an array along the primary fluidic pathway;
such that the flowing sample interacts with one or more biochemical agents present at the one or more detection pads to generate and detect a signal indicative of the presence of the one or more analytes present in the biological sample.

2. The method of claim 1, wherein the test device is a lateral flow assay.

3. The method of claim 1, wherein the configuration of the primary fluidic pathway directs applied biological sample to flow vertically along the primary fluidic pathway.

4. The method of claim 1, wherein the configuration of the primary fluidic pathway directs applied biological sample to flow substantial perpendicular along the primary fluidic pathway.

5. The method of claim 1, wherein the configuration of the primary fluidic pathway directs applied biological sample to flow angularly along the primary fluidic pathway.

6. The method of claim 1, wherein the flowing sample progresses along the primary fluidic pathway in a first direction and subsequently progresses along the laterally directed fluid branches in a second direction transverse to the first direction.

7. The method of claim 1, wherein the flowing sample is divided at each bifurcation node into first and second lateral branches positioned on opposite sides of the primary fluidic pathway.

8. The method of claim 1, wherein each detection pad (304 A-N) receives obtained flowing sample from two laterally opposed branches.

9. The method of claim 1, wherein the signal represents and indicates concentration or level of one or more analytes present in the biological sample.

10. The method of claim 1, wherein the signal is colorimetric, fluorescent, chemiluminescent, radiometric, or electrochemical.

11. The method of claim 1, wherein the biological sample comprises urine, semen, sperm, blood, tears, and saliva.

12. A method for improving quantification of a biological sample in a test device, the method comprising:
(a) applying the biological sample to a sample-introduction region of the test device to obtain a flowing sample along a primary fluidic pathway of the test device;
(b) enabling bifurcation of the obtained flowing sample at one or more bifurcation nodes disposed along the primary fluidic pathway into at least two laterally directed fluid branches to flow towards one or more detection pads arranged in an array along the primary fluidic pathway;
(c) facilitating the obtained flowing sample to pass through at least two successive bifurcation levels prior to reaching the one or more detection pads;
(d) allowing the obtained flowing sample in the at least two laterally directed fluid branches to contact the one or more detection pads, each detection pad of the one or more detection pads, comprising one or more biochemical agents configured to generate and detect signal; and
(e) quantifying the biological sample by measuring the signal generated at the one or more detection pads to obtain an improved quantification output.

13. A test device for detecting one or more analytes in a biological sample, the test device comprising:
(a) a sample-introduction region configured to receive the biological sample;
(b) a primary fluidic pathway extending in a first direction from the sample-introduction region;
(c) one or more bifurcation nodes disposed along the primary fluidic pathway, each bifurcation node being configured to divide a portion of the received biological sample flowing along the primary fluidic pathway into a first and second laterally extending branches positioned on opposite sides of the primary fluidic pathway; and
(d) one or more detection pads arranged in an array along the primary fluidic pathway, each detection pad of the one or more detection pads being fluidically connected to a respective pair of the laterally extending branches and enabling the sample to flow (i) along the primary fluidic pathway, and subsequently (ii) outward and inward toward the one or more detection pads.

14. The test device of claim 13, wherein the test device is a lateral flow assay device.

15. The test device of claim 13, wherein the configuration of the primary fluidic pathway directs the received biological sample to flow vertically along the primary fluidic pathway.

16. The test device of claim 13, wherein the configuration of the primary fluidic pathway directs the received biological sample to flow substantially perpendicular to the primary fluidic pathway.

17. The test device of claim 13, wherein the configuration of the primary fluidic pathway directs received biological sample to flow angularly along the primary fluidic pathway.

18. The test device of claim 13, wherein the array of one or more detection pads (304 A-N) comprises a spaced tiered arrangement along the primary fluidic pathway.

19. The test device of claim 13, wherein each detection pad of the one or more detection pads comprises one or more biochemical agents configured to generate signal that enable quantification of an analyte level across the array of one or more detection pads.

20. The test device of claim 13, wherein each detection pad of the one or more detection pads is positioned along a central axis relative to the pair of laterally extending branches fluidically connected to the array of one or more detection pads.

21. The test device of claim 13, wherein the biological sample is selected from urine, semen, sperm, blood, tears, and saliva.

22. A test device for improving quantification of a biological sample, the test device comprising:
(a) a sample-introduction region configured to receive the biological sample;
(b) a primary fluidic pathway extending in a first direction from the sample-introduction region;
(c) one or more bifurcation nodes disposed along the primary fluidic pathway, each bifurcation node being configured to divide a portion of the received biological sample flowing along the primary fluidic pathway into first and second laterally extending branches positioned on opposite sides of the primary fluidic pathway; and
(d) one or more detection pads arranged in a spaced tiered arrangement along the primary fluidic pathway, each detection pad of the one or more detection pads being fluidically connected to a respective pair of the laterally extending branches and enabling the sample to flow (i) along the primary fluidic pathway, and subsequently (ii) outward and inward toward the one or more detection pads.
